(19) United States
(12) Patent Application Publication
(10) Pub. No.: US 2009/0054979 A1
(43) Pub. Date: Feb. 26, 2009

(54) Phonatory Implant

(75) Inventors: Christian Debry, Paris (FR); Andre Walder, L'Hay les Roses (FR)

Correspondence Address: BUCHANAN, INGERSOLL & ROONEY PC, POST OFFICE BOX 1404, ALEXANDRIA, VA, 22313-1404, US

(73) Assignees: PROTIP SAS, Lingolsheim (FR); Universite Louis Pasteur (ULP), Strasbourg (FR); Hopitaux Universitaires de Strasbourg (HUS), Strasbourg (FR)

(21) Appl. No.: 11/992,189

(22) PCT Filed: Sep. 21, 2006
(86) PCT No.: PCT/FR2006/002156

§ 371 Date: Mar. 18, 2008

(30) Foreign Application Priority Data
Sep. 23, 2005 (FR) 0509730

(51) Int. Cl. A61F 2/20

(52) U.S. Cl. 623/9

(57) ABSTRACT

The invention concerns a phonatory implant (1) consisting of a central portion (3) forming a hollow cylindrical body having at each of its ends a lateral portion (5, 7) forming a shoulder or flange, said implant (1) being urged to be positioned in an opening or fistula running through the tracheoesophageal wall (9), each lateral portion (5, 7) abutting respectively at the inner surfaces of the walls of the trachea (13) and of the esophagus (11) so as to maintain the implant (1) in position, said phonatory implant (1) consisting of at least two parts (A, B), at least one of the two parts being called mobile part (B), and each lateral portion (5, 7) being arranged on a different part of the implant (1) so as to enable the implant to be inserted through an opening of reduced size corresponding to the diameter of the central portion.

The present invention relates to the field of prosthesis or phonatory implants having for function the speech restoration for a patient subject for example to partial or total ablation of trachea and/or larynx. More particularly, the present invention relates to a phonatory implant being urged to be introduced within the tracheoesophageal wall, through an orifice or fistula formed in this wall, so as to enable the air coming from the trachea to reach the mouth. The vocal cords being absent, the patient can therewith modulate sound with his tongue in a comprehensible voice.

There are, to date, several techniques aiming at restoring speech to patients with a laryngotomy. We can mention, as an example, the use of an electronic device or <<electro-larynx>>. Such a device, apart from producing a monotonous and mechanical sound, forces the patient to maintain the device in place at his neck when speaking.

More recently, several devices consisting of prosthesis directly introduced through the tracheoesophageal wall were developed. As non-restrictive examples, the inventions described in the patent applications U.S. Pat. No. 4,911,716 U.S. Pat. No. 4,614,516 U.S. Pat. No. 4,435,853 or U.S. Pat. No. 5,391,205 can be mentioned. Those prosthesis consist of hollow tubes having at their end, in most cases, shoulders or flanges so as to maintain the prosthesis in place through the tracheoesophageal wall. Such prosthesis are generally made of soft material such as silicone, so as to be able to fold or twist one of the ends forming shoulder to enable its introduction through the fistula, said folded or twisted end recovering its initial shape on the other side of the wall. In practice, it is necessary to open a fistula of a size that is higher than the diameter of the prosthesis body so as to enable the insertion of the folded or twisted end, which results, in addition to unnecessarily trauma, in many disadvantages. Actually, once the prosthesis is inserted, the binding between the prosthesis body and the tracheoesophageal wall is very poor because of the interstice resulting from the size difference between the fistula and the prosthesis body. The main disadvantage is that the prosthesis will have a tendency to move and the tissues of the tracheoesophageal wall will hardly heal on the external surface of the prosthesis, all the more so since patients are made vulnerable by the treatments of chemo and/or radiotherapy implemented or added after the ablation of the larynx and vocal cords. Moreover, this poor quality binding is at the origin of many fluid, saliva, water, air leakages, embarrassing for the patient and often leading to the definitive ablation of the prosthesis, with sometimes a need for a surgical act of closing. Another disadvantage comes from the fluid accumulation in this interstice, forcing the prosthesis removal for cleaning or replacement purposes, with all the risks linked to such an operation.

The patent application EP 0651980, describing a prosthesis aiming to limit the fluid or food flow coming from esophagus towards the trachea, can also be mentioned. Therefore, the invention proposes to provide valves or backflow-preventing valves. Nevertheless the disadvantages above mentioned persist. Another improvement is suggested in the patent application WO 96/03095 which describes the use of opaque pigments within the silicone resin used for the prosthesis manufacturing. According to the applicant, the interest is to enable an easier checking of the positioning or the bending of the prosthesis, with X-rays as an example, and thus to avoid any unnecessary replacement. Even if this patent application brings an undeniable advantage, in practice it solves none of the disadvantages detailed above.

Conscious that most of the disadvantages are coming from the necessity to make a fistula in the tracheoesophageal wall of a larger size than the prosthesis diameter, different devices were developed to enable the insertion of the prosthesis while limiting at the most the size of the made orifice. A first alternative is described in the patent application WO 03/057082 and consists of manufacturing the retention flange alternating thickness of the sections, which facilitates the folding of the flange and so enables a diminution of the size, in the folded position, of the end to be inserted. If that improvement is a definite step forward, it is only a partial solution to the problem because it is still necessary to insert within the fistula a folded end that has an appreciably more important size than the body of the prosthesis.

Another very common alternative consists of a device with a capsule or a shell shape, wherein the folded end of the prosthesis is inserted. If the use of such a device really facilitates the prosthesis introduction in the fistula, it does not enable a dramatically reduction of the diameter of the fistula. The patent application EP 0551198 describes such an insertion device with, moreover, the property of being soluble in the fluids present in the esophagus. As stated before, this property facilitates the removal of the device but does not enable to reduce the size of the fistula opened in the tracheoesophageal wall and therefore, does not overcome the disadvantages described above.

Finally, another well-known disadvantage is the fungal colonization, especially by Candida Albicans, of the silicone constituting the prosthesis. Actually, the existence of such an interstice in aerobic, moist and dark conditions, constitutes a favorable area to bacteria or fungi development. Today, a known solution is the application of a local antifungal agent such as Nystatin® or Mycelex®. To be efficient, these antifungal agents need to be applied at least twice a day, which implies manipulations by the patient and thus, not only some discomfort but also some risks of displacement or wrong repositioning of the prosthesis. However, even when combined to antifungal treatments, a silicone prosthesis period of life does not exceed six months, thus forcing its periodical replacement. This surgical intervention repetition multiplies the healing and fistula size increase problems as described above.

Consequently, to date there is a real need for a prosthesis allowing speech restoration and overcoming all the disadvantages described above.

The present invention proposes to overcome all the disadvantages above mentioned and provides a phonatory implant completely new and based on a brand new inventive concept that was never described and even not suggested in the prior art.

The main purpose of the applicant is to provide a phonatory implant having a perfect behavior within the tracheoesophageal wall and whose body is in direct contact with said wall, thus preventing the risks of displacement of the implant, of air leakage or of fluid or food accumulation at the level of the tracheoesophageal wall.

Therefore the applicant developed an implant going against the whole prejudices and current researches that aim to, as described above in the prior art, reduce at maximum the prosthesis rigidity so as to be able to fold up it on itself as much as possible. The present invention investigates another alternative as for the first time, the use of a rigid implant made of several parts is considered.

According to a first embodiment, the present invention consists of a phonatory implant (1) consisting of a central portion (3) forming a hollow cylindrical body having at each of its ends a lateral portion (5, 7) of higher circumference forming a shoulder or flange, said implant (1) being urged to be positioned in an orifice or fistula running through the tracheoesophageal wall (9), each lateral portion (5, 7) abutting respectively at the inner surfaces of the walls of the trachea (13) and of the esophagus (11) so as to maintain the implant (1) in position, said phonatory implant (1) consisting of at least two parts (A, B), at least one of the two parts being called mobile part (B), and each lateral portion (5, 7) being arranged on a different part of the implant (1) so as to enable the implant to be inserted through an orifice of reduced size corresponding to the diameter of the central portion, said mobile part being made interdependent with the central portion using an in situ linking element.

By the term hollow cylindrical body, it must be understood any geometrical form approximately cylindrical, i.e. without any angular form. Any “partly cylindrical” form or any approximately rounded form will have to be considered as an equivalent form and is encompassed within the scope of protection conferred by the present patent application.

In practice, the diameter of the part to be inserted corresponds to the diameter of the central part forming cylindrical body. The shoulder forming stop, corresponding to the second part of the implant, will not have to be inserted within the fistula but is to be positioned once the implant is inserted. Consequently, using the implant according to the present invention, it is not necessary any more to open a fistula of a sufficient size, i.e. higher than the size of the implant body, to be able to introduce one of the implant ends folded on itself, since a fistula of a size corresponding to the real diameter of the implant body is sufficient.

According to a first embodiment wherein the implant is in two parts, the part constituted by the central portion with, at one of its ends, a lateral portion which is introduced within the fistula until the first lateral portion comes to a stop at the level of the tracheoesophageal wall. Then the surgeon has only to fix on the free end, the second part consisting of the second lateral portion.

According to another embodiment of the invention, the implant can be made of several parts like, as a non-restrictive example, in three parts respectively corresponding to the central portion and the two lateral portions. In order to be able to make the different parts of the implant according to the present invention interdependent, linking elements can be considered. Such linking elements will be described hereafter.

However, according to a first preferred embodiment of the invention, the phonatory implant is characterized in that said mobile part (B) consists of a disc directly fixed at the level of the free end (15) of the central portion (3).

According to a second more preferred embodiment, the mobile part (B) of the phonatory implant comprises a hollow cylindrical body (17) having a diameter (d) lower than the diameter (D) of said hollow cylindrical body forming the central portion (3) of the implant (1) so as to be able to slide within said implant.

An advantage of this embodiment is that it can be envisaged to avoid using any linking element. Actually, the mobile part being sized such as to be embedded in the hollow cylindrical body of the implant, the maintenance of the two parts can be made only by the forces of friction between the two parts. Nevertheless it remains better to provide some linking elements in addition.

By the expression “linking element”, it must be understood any mean making possible the maintenance of the mobile part on the fixed part. In a preferred manner, it must be envisaged a non-permanent linking element, i.e. the surgeon as an example, must be able to separate the different parts of the implant.

As non-restrictive examples, linking elements such as screws, nuts or any such mechanical means can be cited. It can also be considered <<clip>> means or electromagnetic means such as magnets of opposite polarity.

More particularly, according to a preferred embodiment, the phonatory implant (1) is characterized in that the linking element of the mobile part (B) to the central portion (3) consists of a female thread (18) and a captive screw bearing a male thread (19) respectively at the level of the free end (15) of the central portion (3) and at the base (23) of the mobile lateral portion (B).

As mentioned above, the implant according to the present invention does particularly well over prior art in that it is made of a rigid material. The use of such a material is new as it goes against the developments to date, as showed by the inventions described in the prior art that tend, on the contrary, to reduce at the maximum the rigidity of the implants (see for example the patent application WO 03/057082).

More particularly, the phonatory implant (1) according to the invention is preferably made of a rigid material that is inert from a biological level.

Such inert materials from a biological level can consist of, as an example, porous glass, coral, bone powder or foam of titanium using titanium hydride.

According to a preferred embodiment, the implant is made up of a biocompatible metallic material, at least in part, preferentially titanium and more preferably microporous titanium.

Microporous titanium is made up of a three-dimensional juxtaposition of plain titanium balls (see standard NF ISO 5832-2 dated March 1997) obtained according to the method described in the patent application EP 0856 299 A1. According to that method, the metallic prosthesis has an open porosity characterized by interspheroid spaces with a dimension approximately equal to one third of the diameter of the powders, i.e. comprised between 50 and 150 mm for powders from 150 to 500 μm.

The method for preparation of the metallic prosthesis according to one of the embodiments described in the patent EP 0 856 299 A1 implies the implementation of the several successive stages as follows:
obtaining by pulverization with a rotating electrode (or any other method giving spheres with low or no bumps) of a powder made up of metallic microspheres of titanium or of titanium based alloy metallic microspheres or of any other biocompatible alloy,
possible gauging of the microspheres by sifting,
forming the prosthesis by heat treatment aiming to interlock said microspheres at the level of their contact areas in an appropriate shaped mould.

As evoked in said patent application, within the context of such a method, the forming of the prosthesis can be done by heat sintering under vacuum or by flash welding in an appropriate shaped mould equipped with electrodes wisely placed through the walls of said moulds. Before and/or during the forming operation, the metallic microspheres can be subject to a vibrating operation allowing a perfect filling of the mould, an optimization of the mechanical properties and a more homogenous porosity through the thickness of the resulting prosthesis.

Another advantage of the present invention, resulting in part from the use of a rigid metallic material, lies in the possibility to use several materials. Particularly, for economical reasons as an example, the microporous titanium can be used only at the level of the implant surfaces directly in contact with the tissues of the tracheoesophageal wall. Actually, the interest of microporous titanium being to enable the tissue colonization, only the surfaces that are in contact with the tissues of the tracheoesophageal wall are concerned.

More preferentially, at least the implants parts that are in contact with the mucous membranes (25) are in microporous titanium.

Obviously this option is not restrictive and it is also envisaged that the whole implant could be made of microporous titanium except the part receiving the linking element when said element requests a thread. For the options using another linking way such as magnetization, the implant could be entirely made of microporous titanium.

According to another embodiment, the implant parts in contact with the mucous membranes have an embossed or grooved surface intended to facilitate the colonization by tissue, therefore the size of the lateral portions (5, 7) can be dramatically reduced, even removed.

The phonatory implant (1) according to another embodiment presents at least one backflow-preventing valve (27) ideally made of a massive titanium strip, and placed at any of the ends of the phonatory implant.

The backflow-preventing valve is intended for, when it is in the closed position to close up the implant in order to prevent the fluids or the food flow from the esophagus towards the trachea, and when it is in the open position to enable the air expired by the lungs to come from the trachea towards the esophagus and also towards the vocal cords.

For a good understanding of the situation, it is specified that the part of the trachea located on the top of the phonatory implant being not functional anymore, the air inspired by the patient circulates towards a meatus made in the trachea approximately facing the implant; the patient closing this meatus with his fingers as an example and expiring the air when he wishes to use its recovered vocal functions.

Practically, it can be considered to have several backflow-preventing valves.

According to another aspect of the invention, a device facilitating the implant insertion is considered. Said device consists of a “cap”, preferably of a shell shape, being urged to be positioned on the end of the implant to be inserted. Contrary to the prior art, the use of such a device is not intended to maintain a folded end in its centre but rather for a function of comfort intended for facilitating the surgeon's work.

Consequently, it is also claimed the insertion device of a phonatory implant (1), characterized in that it consists of a capsule of a shell shape covering the free end (15) of the central portion (3) to be inserted in the fistula, and once the phonatory implant (1) is in place, being intended to be degraded or removed in order to enable the mobile lateral portion (B) to be fixed.

The present invention encompasses also the use of a phonatory implant (1) for restoring speech to a patient after a laryngotomy or a tracheotomy.

It is obvious that such a use is not restricted to the treatment of laryngotomy or tracheotomy, but it can be applied to any treatment requesting a fluids or air flow restoration towards a wall, preferentially at the level of the tracheoesophageal wall.

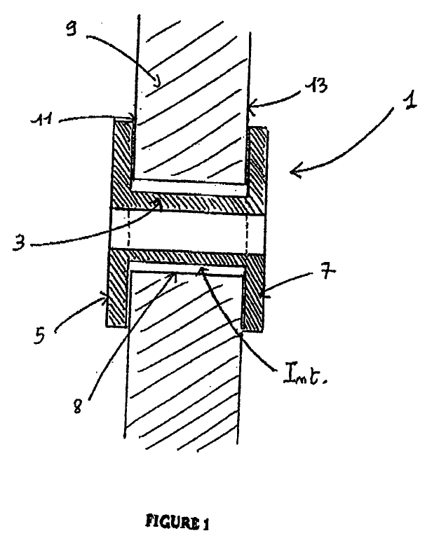

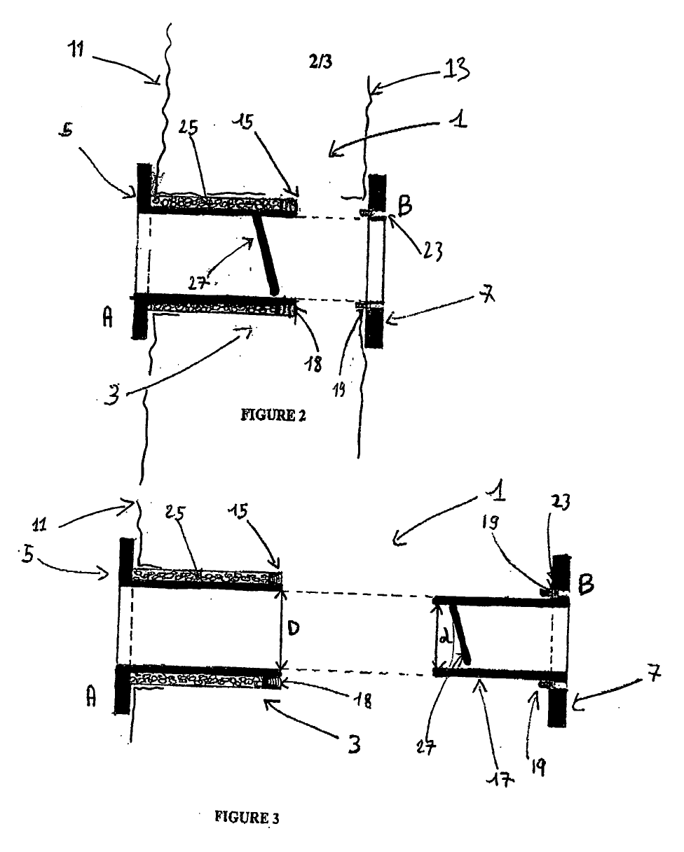

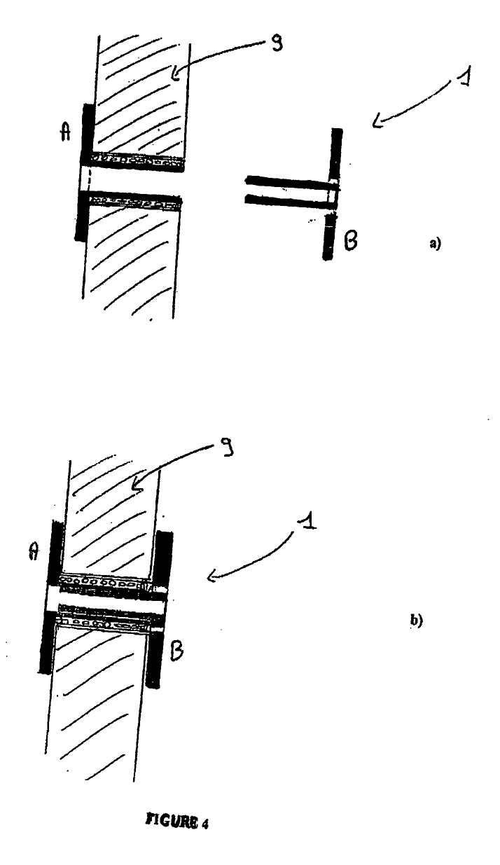

The invention will be better understood, and its advantages will be brought out in a clearer manner, during the following description of two examples of realization, referring to the drawings in appendix wherein:
the FIG. 1 is, out of longitudinal section, a prosthesis according to the prior art,
the FIG. 2 represents an embodiment of the invention out of a longitudinal section,
the FIG. 3 represents another preferred embodiment according to the invention out a longitudinal section, and
the FIG. 4 represents, out of a longitudinal section, an implant according to the invention positioned within the tracheoesophageal wall.

Referring to the FIG. 1, it represents a prosthesis 1 according to the prior art made of a central portion 3 forming a cylindrical body and of two lateral portions 5,7 forming flanges. This prosthesis is made of only one piece and in a flexible material, such as resin or silicone. To be able to be introduced within the tracheoesophageal wall 9, one of its lateral portions 5, 7 had to be folded up on itself in order to reduce its size and to enable its insertion in the fistula. However, as explained above, said fistula has to have a sufficient size to enable the passage of said folded portion 5, 7. With this intention, the fistula has to have a bigger size than the diameter of the central portion 3. The result is an interstice Int. between the central portion 3 and the edges 8 of the fistula made in the wall 9. This interstice Int. is at the origin of leakage and of food and/or fluid accumulation. Moreover, the tracheoesophageal wall tissues cannot come in contact with the prosthesis and the prosthesis is likely to move within the fistula.

If one refers to the FIG. 2, it displays out of a longitudinal section a first non-restrictive embodiment of the invention.

More particularly, it is represented a phonatory implant 1 according to the invention, made of a first part A comprising a central portion 3 and a lateral portion 5 and a second, disc shaped, part B forming the lateral portion 7.

The part B, called mobile part, can be made interdependent of part A using one or several linking element(s). As described above, such linking elements can consist in any mean enabling the fixation and the immobilization of the part B on the part A in a non-definitive manner, i.e. a surgeon as an example, will be able easily to separate them. As detailed above, a thread system, a magnets system, a “clip” system, or any equivalent system can be mentioned.

The FIG. 3 represents a second preferred embodiment according to the invention. The implant 1 is made of a first part A comprising a central portion 3 and a lateral portion 5, and a second part B comprising a central portion 17 forming a hollow cylindrical body and a lateral portion 7. The central portion 17 of part B is dimensioned so as to be able to slide within the central portion 3 of the part A. Therefore, the central portion 17 forming a hollow cylindrical body has a diameter d lower than the diameter D of the central portion 3. Practically, the part B fits into the part A.

In the embodiment represented here, it is also envisaged linking elements (18, 19). However, considering that the two parts, respectively A and B, encase one in the other, the linking elements could be absent; the maintenance resulting from the fitting of the part B in the part A. Whether in the FIG. 2 or in the FIG. 3, a backflow-preventing valve 27 is also represented. This valve acts to enable the passage of air from the trachea towards the oesophagus and to prevent the passage of food from the oesophagus towards the trachea. Of course, said valve 27 could be placed differently, at the level of anyone of part A or B. Moreover, using several valves could also be considered.

Finally, these figures also show a particular feature, namely the disposition of the different materials used. Actually, as described above, the implant of the invention is made of a rigid material, preferentially a metallic material and more preferentially titanium. According to an innovative feature, the parts of the implant that are in contact with the tissues of the tracheoesophageal wall are made of a material that is not only biocompatible, but also that can be colonized, such as microporous titanium. That feature is clearly illustrated by FIGS. 2 and 3 that represent, in black, the parts of the implant that are not in contact with the tissues surrounding the fistula and, in circles, the parts 25 that are in contact with the tissues of the tracheoesophageal wall and that are made, as a consequence, of microporous titanium. Obviously, any material equivalent to microporous titanium, i.e. having similar features of biocompatibility and colonization, could be considered and therefore would have to be treated as an equivalent mean.

The FIG. 4 represents the use of the implant according to the invention. The FIG. 4a represents the part A comprising the central portion 3 inserted within the fistula until the lateral part 5, forming a shoulder, comes in contact with the side 11 on the esophageal side of the tracheoesophageal wall 9. As it can be seen, the external surface 25 of the central portion 3 in contact with the tissues of the wall 9 is made of microporous titanium. The dimensions of the central portion 3 forming the body of the implant are established so as to avoid a space between the external surface of the implant and the tracheoesophageal wall. Then, the part B is introduced by the surgeon and is positioned so as to fit together with part A. The FIG. 4b represents the implant once installed, i.e. once the part B is interdependent of the part A.

Obviously, the different parts of the implant according to the invention are proportioned in order to enable a sufficient flow of air. As a non-restrictive example, the internal diameter d of the part B is preferentially ranging between 3 and 7 mm, and preferentially between 5 and 6 mm. The diameter D corresponding to the level of part A has to have a size enabling the passage of the part A and preferably, has to have a space ranging from 0.05 to 0.3 mm.

The present description consists of a general form of the invention and any modification or obvious improvement, based on the invention principle, has to be considered as being equivalent.

1. A phonatory implant (1) consisting of a central portion (3) forming a hollow cylindrical body having at each of its ends a lateral portion (5, 7) of higher circumference forming a shoulder or flange, said implant (1) being urge to be positioned in an orifice or fistula running through the tracheoesophageal wall (9), each lateral portion (5, 7) abutting respectively at the inner surfaces of the walls of the trachea (13) and of the esophagus (11) so as to maintain the implant (1) in position, and consisting of at least two parts (A, B), at least one of the two parts being called mobile part (B), and in which each lateral portions (5, 7) is arranged on a different part of the implant (1) so as to enable the implant to be inserted through an orifice of reduced size corresponding to the diameter of the central portion, said mobile part being made interdependent with the central portion using an in situ linking element, said phonatory implant (1) being characterized in that the parts of the implant (1) in contact with the mucous membranes (25) are made of micro-porous titanium.

2. The phonatory implant (1) according to claim 1, characterized in that said mobile part (B) consists of a disk directly fixed at the level of the free end (15) of the central portion (3).

3. The phonatory implant (1) according to claim 1, characterized in that said mobile part (B) comprises a hollow cylindrical body (17) having a diameter (d) lower than the diameter (D) of the hollow cylindrical body forming the central portion (3) of the implant (1) so as to be able to slide within the latter.

4. The phonatory implant (1) according to claim 1, characterized in that said linking element being of the mobile part (B) to the central portion (3) consists of a female thread (18) and a captive threaded screw bearing a male thread (19) respectively at the level of the free end (15) of the central portion (3) and at the base (23) of the mobile lateral portion (B).

5. The phonatory implant (1) according to claim 1, characterized in that it is made of a rigid material that is inert at a biological level.

6. The phonatory implant (1) according to claim 5, characterized in that is, at least in part, made of a metallic biocompatible material, and preferentially titanium.

7. The phonatory implant (1) according to claim 1, characterized in that the parts of the implant in contact with the tissues has a embossed or grooved surface intended to facilitate the colonization by tissues.

8. The phonatory implant (1) according to claim 1, characterized in that it presents at least one backflow-preventing valve (27) ideally made of a massive titanium strip.

9. A device for the insertion of a phonatory implant (1) according to claim 1, characterized in that said device consists of a shell-shaped capsule covering the free end (15) of the central portion (3) to be inserted within the fistula and, once the phonatory implant (1) is in place, intended to be degraded or to be removed in order to enable the lateral mobile portion (B) to fix itself.

10. A method for restoring speech to a patient after a laryngotomy or a tracheotomy, comprising introducing a phonatory implant (1) according to claim 1 into the patient.
